(19) United States
(12) Patent Application Publication
(10) Pub. No.: US 2015/0011877 A1
(43) Pub. Date: Jan. 8, 2015

(54) Intra-Operative Fiducial System and Method for Neuronavigation

(71) Applicant: Adventist Health System/Sunbelt, Inc., Altamonte Springs, FL (US)

(72) Inventor: James Edmund Baumgartner, Winter Park, FL (US)

(21) Appl. No.: 14/301,501

(22) Filed: Jun. 11, 2014

Related U.S. Application Data
(60) Provisional application No. 61/833,576, filed on Jun. 11, 2013.
(60) Provisional application No. 61/949,435, filed on Mar. 7, 2014.
(60) Provisional application No. 61/949,421, filed on Mar. 7, 2014.

(51) Int. Cl. A61B 19/00; A61M 5/00

(52) U.S. Cl. CPC A61B 19/54; A61M 5/007; A61B 2019/5454; A61B 2019/5466
USPC 600/431

(57) ABSTRACT

An imageable fiducial marker includes a base formed from a material transparent to radiation from an imaging source and a pair of fiducial elements fixed to the base at a preselected separation. A bracket temporarily secures the marker to a skull of a human for intra-operative scanning in neurosurgery procedures, wherein only three fiducial pairs provide for reliable surgical navigation.

### CROSS-REFERENCE TO RELATED APPLICATIONS

This application claims priority to U.S. Provisional Application Ser. No. 61/833,576, filed Jun. 11, 2013, entitled “Intra-Operative Fiducial System and Method for Neuronavigation,” U.S. Provisional Application Ser. No. 61/949,421, filed Mar. 7, 2014, entitled “Probe for Surgical Navigation of the Brain,” and U.S. Provisional Application Ser. No. 61/949,435, filed Mar. 7, 2014, entitled “MRI-Compatible Fiducial Markers and Methods for Using the Same,” the disclosures of which are herein incorporated by reference in their entirety, and are all commonly owned.

### FIELD OF INVENTION

Embodiments of the invention generally relate to fiducial markers placed in a field of view of an imaging system for providing a reference point, and in particular to fiducial markers temporarily fixed to a patient during intra-operative imaging, such as with MRI or CT scans.

### BACKGROUND

As is well known in the medical arts, a fiducial marker or fiducial is an object placed in the field of view of an imaging system, which appears in the image produced, for use as a point of reference or a measure. The fiducial is typically placed into or on the imaging subject, or a mark or set of marks in the reticle of an optical instrument. Fiduciary markers are well known to be used in a wide range of medical imaging applications.

By way of example, images of the same subject produced with different imaging systems may be correlated by placing a fiduciary marker in the area imaged by the different systems. As a result, functional information or positron emission tomography may be related to anatomical information provided by magnetic resonance imaging (MRI), by way of example. Further, fiducial points established during an MRI can be correlated with brain images generated by magnetoencephalography (MEG) to localize the source of brain activity. Such fiducial markers are often created in the MRI and computed tomography images.

Measurement and recording techniques which are not primarily designed to produce images, such as the MEG, electroencephalography (EEG), electrocardiography (EKG), and others, produce data typically represented as maps containing positional information. Imaging systems such as computed tomographic (CT) x-ray imagers, positron emission tomographic (PET) scanners, single photon emission computed tomography (SPECT) scanners and nuclear magnetic resonance imaging (MRI) machines have provided an ability to improve visualization of the anatomical structure of the human body.

A problem associated with such scanning techniques concerns the accurate selection and comparison of views of identical areas in images that have been obtained essentially at the same time using different image modalities, e.g., CT, MRI, SPECT, and PET. In order to relate the information in an image of the anatomy to the anatomy itself, it is necessary to establish a one-to-one mapping between points in the image and points of anatomy for registering image space to physical space. Further, the registration of one image space onto another image space may require multiple imaging steps, which during intra-operative procedures is undesirable. A goal of registering two arbitrarily oriented three dimensional images is to align the coordinate systems of the two images such that any given point in the scanned anatomy is assigned identical addresses in both images. The fiducials are used to correlate image space to physical space and to correlate one image space to another image space. The fiducial markers provide a constant frame of reference visible in a given imaging mode to make registration possible.

Well known methods of using fiducial markers to obtain registration of image data across time is described, by way of example, in U.S. Pat. Nos. 4,991,579 and 5,142,930 which address implanting the fiducials within a patient using a series of three fiducial markers whose location can be determined in the image space of an imager. Image markers can be temporary or permanent with respect to the duration of their placement within the human body. Typically, permanent markers are placed entirely beneath the epidermis of the skin for extended periods of time. Temporary markers may include a base that is implanted into bone, and a temporary image marker portion that is attached to the base for brief intervals of time. In both the temporary and the permanent markers, the marker may use aqueous imaging agents to provide imaging capability in the desired imaging modality or modalities. The precise location of a geometric center of the marker must be determined.

By way of example, U.S. Pat. No. 4,612,930 discloses a head fixation apparatus including a crown and skull pins, wherein the crown surrounds the head of a patient and is held by the skull pins. U.S. Pat. No. 4,991,579 describes a fiducial implant for the human body that is configured to be detected by an imaging system when placed beneath the skin. As above described, the placement of three fiducial implants into a portion of the anatomy of the human body allows for the recreation of a particular image slice of the portion of the anatomy taken by an imaging system with respect to one time period and at subsequent imaging sessions with different scan modalities. This provides a doctor with the ability to accurately follow the progress of the portion of the anatomy of interest. The three fiducial implants allow a target to be identified within the portion of anatomy relative to an external coordinate system.

There remains a need for a temporarily implanted fiducial that allows use of an intra-operative MRI scanner with neuro-navigation, especially in children below the age of five. With sterile MRI or CT compatible fiducials, a desirable use of intra-operative navigation results. A safer and improved accuracy is desirable for surgery on children with intra-cranial disease. Embodiments of the invention herein described are directed to providing such a solution.

### SUMMARY

One embodiment of the present invention may comprise a fiducial marker pair having one marker in a fixed space-relationship to a second marker. The fiducial marker pair may be temporarily held in a fixed location in an anatomy during an intra-operative scanning procedure. A bracket may be employed to attach the marker pair to a skull of a patient, by way of example. Optionally, absorbable plates may be used to attach the fiducial marker pair to the skull.

One embodiment may be dimensioned for pediatric neurosurgeons requiring precision in locating surgical points without having to execute intra-operative scanning multiple times.

One fiducial marker according to the teachings of the present invention may comprise a pure gold sphere incorporated in a method for cranial and spinal applications. The fiducial marker pair comprising at least two spheres may be carried by a housing or bracket. It is well known to use a small hole in the bone, wherein the sphere is placed and the hole sealed with bone wax to avoid marker migration in a permanent use of the fiducial. However, it is desirable to use such spheres in a temporary fiducial marker, as provided by the teachings of the present invention.

As above described, implantable fiducial markers provide a highly effective method of ensuring accurate target localization for tumors or organs which move in respect to external anatomy, as well as for a precise localization needed for stereotactic rating of therapy. A variety of fiducial markers are known and typically use in surgery related to the prostate, lungs and abdomen, breasts, and head and neck as will herein be described by way of example, a bone marker formed from a pure gold sphere approximately 2 mm is desirable for head and neck. Embodiments of the present invention are directed to a temporary fixation of such fiducial marking procedures.

### BRIEF DESCRIPTION OF THE DRAWINGS

Embodiments of the invention are described by way of example with reference to the accompanying drawings in which:

FIG. 1 is a perspective view of one embodiment of the invention illustrating a fiducial element pair carried within a base formed of a plastic material transparent to imaging source radiation, wherein the base is operative with a bracket and screws for temporarily securing the fiducial element pair to a skull, wherein each fiducial element comprises a spherical shape;

FIGS. 2A, 2B and 2C are perspective views of alternate embodiments of a base carrying the fiducial element pair;

FIG. 3 is a perspective view of an alternate embodiment illustrating cylindrical shaped fiducial elements carried within a bracket;

FIG. 3A is a fiducial element pair illustrated in FIG. 3;

FIG. 4 is a diagrammatical illustration of a fiducial marking system including three pairs of fiducial elements;

FIG. 5A and 5B are top plan and side views, respectively, of a fiducial marker comprising a base formed as a disk and a fiducial marker element pair carried on a surface of the disk; and

FIG. 6A, 6B and 6C are exploded perspective, top plan and side views, respectively, of a fiducial marker comprising a base formed as a disk and an alternate fiducial marker element pair carried on a surface of the disk, wherein the disk and marker element pair are to be secured to a skull using screws, by way of example.

### DETAILED DESCRIPTION OF EMBODIMENTS

The present invention will now be described more fully hereinafter with reference to the accompanying drawings, in which embodiments of the invention are shown by way of illustration and example. This invention may, however, be embodied in many forms and should not be construed as limited to the embodiments set forth herein. Rather, these embodiments are provided so that this disclosure will be thorough and complete, and will fully convey the scope of the invention to those skilled in the art.

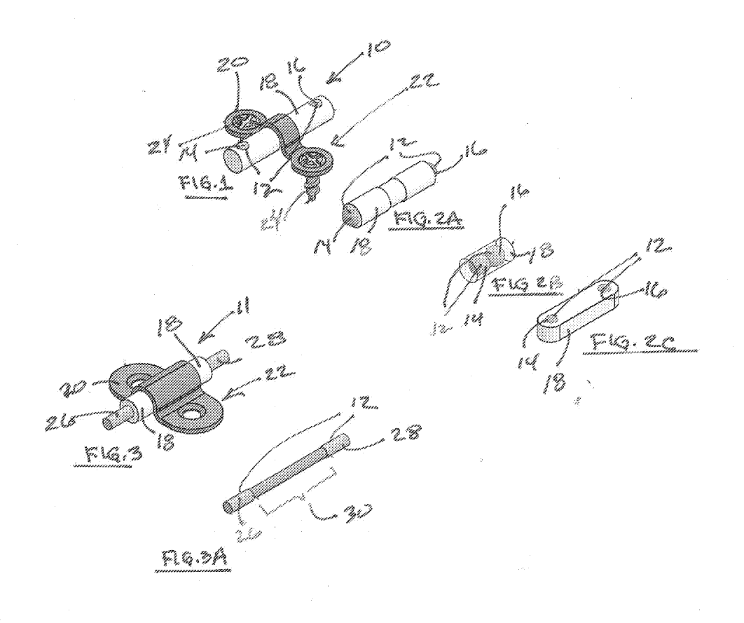

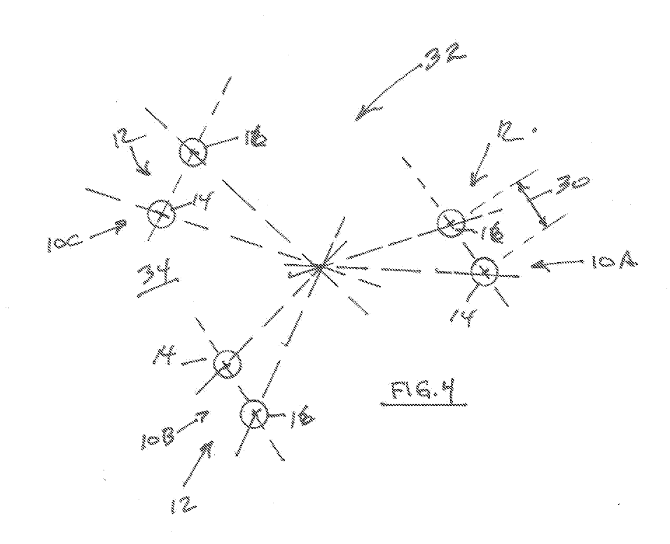

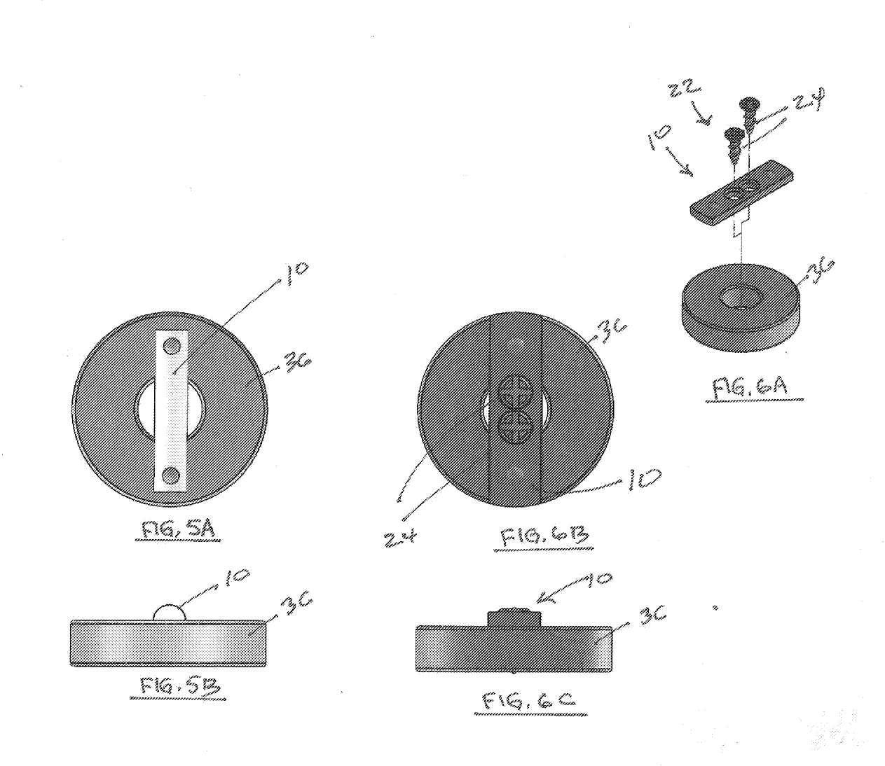

As illustrated initially with reference to FIG. 1, one fiducial marker 10 according to the teachings of the present invention comprises a fiducial element pair 12 comprising fiducial elements formed as spheres 14, 16 fixed to a base 18, herein described as a casing. The casing 18 is dimensioned to be temporarily secured to a skull, by way of example, using a bracket 20 having securing means 22, such as screws 24 herein illustrated. The casing 18 is transparent to imaging modalities to be employed. By way of example for an MRI or CT scan, the casing 18 may be formed from a plastic material. The spherical fiducial elements 14, 16, herein illustrated as gold spheres, may be embedded in an injection molded plastic casing 18, as further illustrated with reference to FIGS. 2A, 2B and 2C. The bracket 20 and securing means 22 may or may not be transparent to scanning radiation, as desired. By way of example, the securing means 22 may comprise titanium screws 24, as herein illustrated passing through holes in end portions of the bracket 20.

By way of further example and with reference to FIGS. 3 and 3A, the fiducial marker 10 may comprise cylindrically shaped elements. Whether a gold sphere marker 10, or cylindrical fiducial marker 11, or the like is employed, the fiducial elements 14, 16 or 26, 28 are secured at a fixed separation distance 30 between markers elements within the marker pair 12.

As illustrated with reference to FIG. 4, one embodiment of a fiducial marking system 32, according to the teachings of the present invention, comprises three fiducial markers 10A, 10B, 10C temporarily fixed to a skull 34 during an intraoperative scanning procedure, by way of example. By way of further example and as illustrated with reference to FIG. 4, the three fiducial marker pairs 12, unlike typical fiducial markers using a single element, improves on identifying mapping locations and avoid the need for multiple measurements. As a result, improved reliability and efficiency are realized over typical fiducial employment.

Optionally, and as illustrated with reference to FIGS. 5A and 5B, the bracket 20 may comprise a disk 36 formed from a material transparent to the scanning modality and securing the fiducial marker 10 having the element pair 12 secured with the base 18. The disk 36, as herein described by way of example, may comprise the fiducial element pair 12 and used as a multi-locality fiducial system 32 used in image guided surgery to correlate scanned images to a patient anatomy.

Further, the use of multiple fiducial marker pairs 12 is beneficial for both CT and MRI use optionally, wherein each fiducial marker pair may be attached to a stick on a base (such as that illustrated with reference to FIG. 5A) which may be used for routine stereotactic planning, by way of example.

Although the invention has been described relative to various selected embodiments herein presented by way of example, there are numerous variations and modifications that will be readily apparent to those skilled in the art in light of the above teachings. It is therefore to be understood that, within the scope of the claims supported by this specification, the invention may be practiced other than as specifically described.

That which is claimed is:

1. An imageable fiducial marker comprising:
a base formed from a material transparent to radiation from an imaging source; and
first and second fiducial elements fixed to the base at a preselected separation.

2. The imageable fiducial marker according to claim 1, further comprising:
a bracket dimensioned for receiving the base; and
means for removably securing the bracket to a skull of a human.

3. The imageable fiducial marker according to claim 1, wherein the base is formed from a plastic material.

4. The imageable fiducial marker according to claim 1, wherein the first and second fiducial elements are molded within the base.

5. The imageable fiducial marker according to claim 1, wherein at least one of the first and second fiducial elements comprises at least one of a spherical shape and a cylindrical shape.

6. The imageable fiducial marker according to claim 1, wherein at least one of the first and second fiducial elements is gold.

7. The imageable fiducial marker according to claim 1, wherein the base comprises a cylindrical body, and wherein the fiducial elements are secured within the cylindrical body.

8. The imageable fiducial marker according to claim 7, further comprising a bracket having a central portion formed for receiving the cylindrical body and spaced apart end portions including means for removably securing the bracket to a skull of a human.

9. The imageable fiducial marker according to claim 8, wherein the securing means comprises an opening extending through each of the end portions and a screw extending through the opening for penetrating the skull and securing the marker thereto.

10. The imageable fiducial marker according to claim 9, wherein the screw is formed from a titanium material.

11. An intra-operative neuro-navigation imaging system comprising:
a base formed from a material transparent to radiation from an imaging source;
first and second fiducial elements fixed to the base at a preselected separation from each other;
a bracket dimensioned for receiving the base; and
means for removably securing the bracket to a skull of a human.

12. The system according to claim 11, wherein the first and second fiducial elements together form a fiducial pair, and wherein the system comprises only three fiducial pairs.

13. The system according to claim 11, wherein the first and second fiducial elements together form a fiducial pair, and wherein the system comprises at least three fiducial pairs.

14. The system according to claim 11, wherein the first and second fiducial elements together form a fiducial pair, and wherein each element comprises a similar shape and similar composition.

15. The system according to claim 14, wherein the shape is at least one of spherical and cylindrical, and wherein the composition comprises a gold material.

16. The system according to claim 11, wherein the first and second fiducial elements are molded within the base.

17. The system according to claim 11, wherein the first and second fiducial elements each have a cylindrical shape, and wherein each is fixed to an end of a cylindrical rod, the rod being formed of a material transparent to the imaging source of radiation.

18. The system according to claim 17, wherein the rod is carried by at least one of the base and the bracket.

19. The system according to claim 11, wherein the fiducial elements comprise gold spheres having about a 2 mm diameter.

20. The system according to claim 11, wherein the base comprises a disk having an opening extending therethrough, the opening sufficient for receiving the securing means extending therethrough.

21. The system according to claim 19, wherein the base comprises a plate.
